(19) United States
(12) Patent Application Publication
(10) Pub. No.: US 2008/0081377 A1
(43) Pub. Date: Apr. 3, 2008

(54) NUCLEIC ACID DETECTION METHOD AND EXAMINATION KIT

(75) Inventor: Masaaki Kobayashi, Kawasaki-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/852,843

(22) Filed: Sep. 10, 2007

(30) Foreign Application Priority Data
Sep. 29, 2006 (JP) 2006-266538

(51) Int. Cl. G01N 33/00; C07H 21/00

(52) U.S. Cl. 436/94; 536/24.3

(57) ABSTRACT

Nucleic acid detection method for detecting a target nucleic acid in an analyte solution, including the steps of preparing a mixed solution by mixing, labeled nucleic acid probes in which nucleic acid probes to be associated with the target nucleic acid are immobilized on microcarriers, the analyte solution; and a nucleic acid-aggregating agent, and detecting the presence or absence or degree of aggregation of the microcarriers in the mixed solution.

### BACKGROUND OF THE INVENTION

1. Field of the Invention

The present invention relates to a nucleic acid detection method and an examination kit.

2. Description of the Related Art

Base sequence analysis has been carried out actively on the genomes of various organisms including humans. As a result, diagnosis, decision about treatment courses, post-treatment management and prognostic expectation have been practiced for diseases including hereditary disease, cancer, infectious disease and adult disease through the detection of target nucleic acids such as genes, DNA and RNA.

Genetic diagnosis, which uses the aggregation of complexes of latex microparticles or colloidal metal particles with DNAs, has received attention as a technique used in target gene detection.

Japanese Patent Application Laid-Open No. H10-117798 has disclosed the detection of the presence or absence of a target nucleic acid in an analyte sample using a substance (aggregation promoter capable of binding to genes) having both the property of intercalating with nucleic acids and the property of aggregating particles such as colloidal silver. A sample is subjected in advance to treatment for amplifying only a target nucleic acid, for example, by a PCR (polymerase chain reaction) method. Aggregation promoters capable of binding to genes and colloidal silver particles are added to the sample. When the target nucleic acid is present in the sample, almost all of the aggregation promoters capable of binding to genes bind to the target nucleic acids amplified by PCR. As a result, the colloidal silver particles are not aggregated. On the other hand, when the target nucleic acid is absent in the sample, nucleic acid amplification does not occur in the sample. Thus, few aggregation promoters capable of binding to genes bind to the nucleic acid in the sample. As a result, the colloidal silver particles are aggregated. As disclosed in Japanese Patent Application Laid-Open No. 10-117798, the presence or absence of such aggregation of the colloidal silver particles can be confirmed, for example, by visual observation, to thereby determine the presence or absence of the target nucleic acid in the sample.

However, the aggregation promoter capable of binding to genes used in the invention described in Japanese Patent Application Laid-Open No. H10-117798 has the property of binding specifically to nucleic acids, for example, the property of distinguishing between single-strand and double-strand nucleic acids, but cannot recognize the base sequence of a target nucleic acid for binding. Therefore, the sequence-specific detection of a target nucleic acid using this invention requires amplifying only the target nucleic acid in advance by a gene amplification method such as PCR. Thus, the invention described in Japanese Patent Application Laid-Open No. H10-117798 must use the gene amplification method. Therefore, this method is difficult to apply to the detection of plural genes and the detection of a target nucleic acid in a sample containing many genes other than the target nucleic acid.

Alternatively, Japanese Patent Application Laid-Open No. 2004-275187 has disclosed the detection of the presence or absence of a target gene using the aggregating property of colloidal gold particles. Specifically, the colloidal gold particles are aggregated when single-strand DNA probes immobilized on the colloidal gold particles form complementary strands with target DNA through association reaction.

However, in the invention described in Japanese Patent Application Laid-Open No. 2004-275187, the colloidal gold particles are aggregated when the target DNA has the same length as that of the DNA of the DNA probe and forms completely complementary strands therewith and when the target DNA has the same length as that of the nucleic acid of the DNA probe and has internal deletion. Thus, the discrimination between target DNA and non-target DNA by aggregation requires designing a DNA probe sequence so that base substitution is located on the free end of the DNA probe (see Table 1 of Japanese Patent Application Laid-Open No. 2004-275187). Furthermore, the invention described in Japanese Patent Application Laid-Open No. 2004-275187 requires adjusting the length of the target DNA in the sample. Therefore, this method requires many preparations such as the purification of amplification products after gene amplification reaction, primer extension reaction and the association reaction of a second nucleic acid probe and is therefore complicated.

### SUMMARY OF THE INVENTION

The present invention enables sequence-specific detection of a target nucleic acid by the convenient preparation of the target nucleic acid and a simple detection method including visual observation.

The present invention provides a nucleic acid detection method for detecting a target nucleic acid in an analyte solution, including the steps of: preparing a mixed solution by mixing: labeled nucleic acid probes in which nucleic acid probes to be associated with the target nucleic acid are immobilized on microcarriers; and the analyte solution; adding a nucleic acid-aggregating agent to the mixed solution; and detecting the presence or absence or degree of aggregation of the microcarriers in the mixed solution.

It is preferred that the above step of preparing a mixed solution by mixing: labeled nucleic acid probes in which nucleic acid probes to be associated with the target nucleic acid are immobilized on microcarriers; the analyte solution; and a nucleic acid-aggregating agent is a step of preparing a mixed solution by mixing the labeled nucleic acid probes with the analyte solution and then adding the nucleic acid-aggregating agent thereto.

Also, it is preferred that the above step of preparing a mixed solution by mixing: labeled nucleic acid probes in which nucleic acid probes to be associated with the target nucleic acid are immobilized on microcarriers; the analyte solution; and a nucleic acid-aggregating agent is a step of simultaneously mixing the labeled nucleic acid probes, the analyte solution and the nucleic acid-aggregating agent

The nucleic acid probes are preferably peptide nucleic acids.

The step of detecting the presence or absence or degree of aggregation of the microcarriers is preferably performed by measuring a color change of the mixed solution.

The nucleic acid-aggregating agent is preferably a substance that does not bind to the nucleic acid probe itself and binds to an associate between the target nucleic acid and the labeled nucleic acid probe.

Another embodiment of the present invention provides an examination kit for detecting a target nucleic acid in a sample, including at least one examination reagent, the one examination reagent including labeled nucleic acid probes in which nucleic acid probes are immobilized on microcarriers.

The examination kit is preferred further to include an examination reagent including a nucleic acid-aggregating agent.

A further embodiment of the present invention provides a nucleic acid detection method for detecting a target nucleic acid in a sample, including the steps of: forming an associate between labeled nucleic acid probes in which nucleic acid probes are immobilized on microcarriers and the target nucleic acid; reacting the associate with a nucleic acid-aggregating agent; and detecting the aggregated state of the microcarriers.

Peptide nucleic acids (PNAs) are preferably used as the nucleic acid probes.

The aggregated state of the microcarriers is preferably detected by measuring a color change of the solution.

A substance that does not bind to the nucleic acid probe itself and binds to the associate is preferably used as the nucleic acid-aggregating agent.

Further features of the present invention will become apparent from the following description of exemplary embodiments with reference to the attached drawings.

### BRIEF DESCRIPTION OF THE DRAWINGS

FIGS. 1A, 1B, 1C and 1D are flow charts illustrating an examination method according to one embodiment of the present invention.

### DESCRIPTION OF THE EMBODIMENTS

A first embodiment of the present invention provides a nucleic acid detection method for detecting a target nucleic acid in an analyte solution, including the steps of: preparing a mixed solution by mixing: labeled nucleic acid probes in which nucleic acid probes to be associated with the target nucleic acid are immobilized on microcarriers; the analyte solution; and a nucleic acid-aggregating agent; and detecting the presence or absence or degree of aggregation of the microcarriers in the mixed solution.

When the microcarriers were confirmed to be aggregated, the target nucleic acid is present in the analyte solution. This indicates that the target nucleic acid is present in the sample.

These steps are usually performed in a solution. In general, the mixed solution is prepared in the following order: a sample solution is mixed with a solution containing labeled nucleic acid probes to form a mixed solution; then, a nucleic acid-aggregating agent or a solution containing a nucleic acid-aggregating agent is added to this mixed solution.

As shown in an example described later, the reaction of the nucleic acid-aggregating agent with the nucleic acid may be faster significantly than the reaction of the nucleic acid-aggregating agent with an associate between the target nucleic acid and the labeled nucleic acid probe. In this case, the nucleic acid probes are mixed with the analyte solution to sufficiently produce the association between the nucleic acid probes and the target nucleic acid. Then, the nucleic acid-aggregating agent should be added to the solution containing these associates. On the other hand, the reaction of the unassociated target nucleic acid with the aggregating agent may not matter, for example in case that these reactions may hardly differ in speed. In this case, a possible method includes simultaneously or almost simultaneously adding the labeled nucleic acid probes and the nucleic acid-aggregating agent to the sample.

The outline of one embodiment of a measurement method according to the present invention will be described with reference to the drawings. The detection of the present embodiment can be practiced according to procedures described below. However, the present invention is not intended to be limited to the procedures.

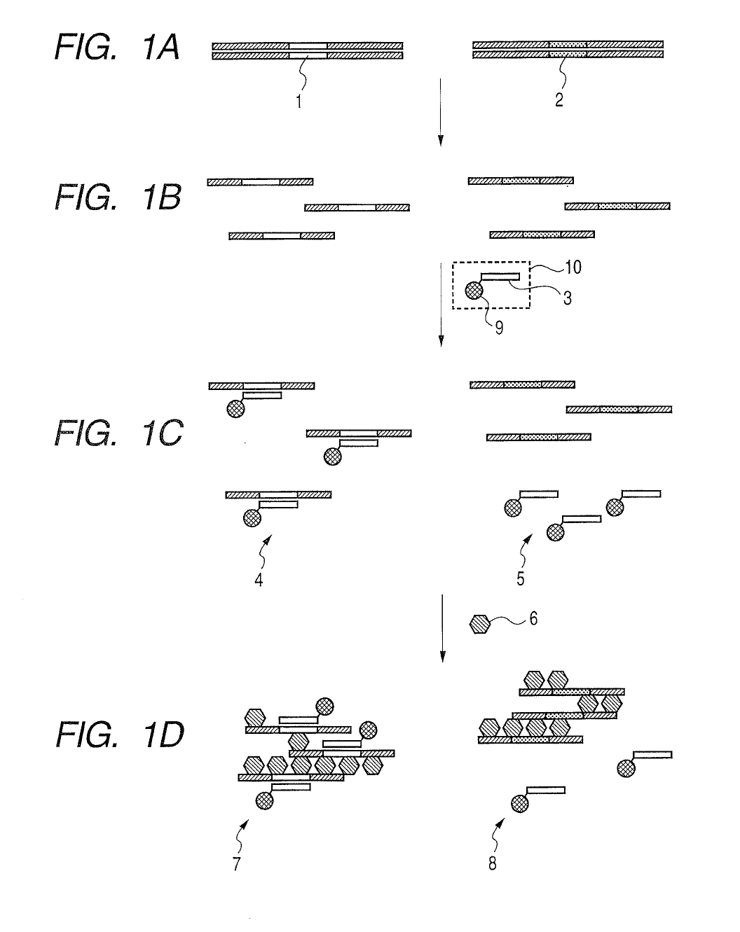

FIGS. 1A to 1D illustrate a detection method according to the present embodiment. An example illustrated on the left side of this drawing is detection using a nucleic acid (target nucleic acid 1) having a sequence complementary to that of a nucleic acid probe 3. An example illustrated on the right side thereof is detection using a nucleic acid (non-target nucleic acid 2) free from a sequence complementary to that of the nucleic acid probe 3.

As illustrated in FIG. 1A, an analyte solution containing target nucleic acids 1 and an analyte solution containing non-target nucleic acids 2 but no target nucleic acids 1 are first prepared. Next, as illustrated in FIG. 1B, the target nucleic acids 1 and the non-target nucleic acids 2 are converted into their respective single-strand nucleic acids by use of gene amplification reaction, primer extension reaction and denature reaction. Then, the nucleic acid probes 3 immobilized on microcarriers 9 (hereinafter, referred to as “labeled nucleic acid probes 10”) are added to the respective analyte solutions to prepare mixed solutions. Association reaction is allowed to occur between the probes and the nucleic acids. As illustrated in FIG. 1C, the target nucleic acid 1 having a sequence complementary to that of the nucleic acid probe 3 is associated with the nucleic acid probe 3 by the association reaction and forms an associated state 4, that is, forms an associate. By contrast, the non-target nucleic acid 2 free from a sequence complementary to that of the nucleic acid probe 3 is not associated with the nucleic acid probe 3 (non-associated state 5). Then, a nucleic acid-aggregating agent 6 is added to the respective solutions to prepare mixed solutions. The nucleic acid-aggregating agent 6 has the property of binding to single-strand and double-strand nucleic acids in an analyte solution and aggregating the nucleic acids. Therefore, when the nucleic acid-aggregating agent 6 is added to the solution forming the associated state 4, the target nucleic acids are aggregated in a state involving the labeled-nucleic acid probes 10 (aggregation 7 of labeled-nucleic acid probes). By contrast, when the nucleic acid-aggregating agent 6 is added to the mixed solution in the non-associated state 5, only the non-target nucleic acids 2 are aggregated by the nucleic acid-aggregating agent 6 and the labeled nucleic acid probes 10 are not aggregated (non-aggregation 8 of labeled nucleic acid probes). In this context, “association” described in the present invention means that a few identical molecular species form an associate through binding by relatively week force such as intermolecular force or a hydrogen bond and behave as like one molecule. Moreover, “aggregation” refers to a phenomenon in which many molecules gather together by the attraction among them and serve as a large assembly.

Specifically, in the present embodiment, when the target nucleic acid 1 is contained in the sample (in other words, when the target nucleic acid 1 is contained in the analyte solution), the microcarriers 9 are aggregated. When the target nucleic acid 1 is not contained in the sample, the microcarriers 9 are not aggregated. Then, the presence or absence or degree of aggregation (in other words, the aggregated state) of the microcarriers 9 can be detected easily, for example, by visual observation or with an aggregated state detector such as an absorbance measurement apparatus.

At the step of detecting the presence or absence or degree of aggregation of the microcarriers, it is preferable that the presence or absence of aggregation of the microcarriers can be detected conveniently by measurement using visual observation. On the other hand, the degree of aggregation can be detected using a method including measuring a color change of the mixed solution based on absorbance determined with a spectrophotometer or a method including detecting the size of the aggregates precipitated under standing or the density or size of the voids therein with an aggregated state detector. Such approaches enable accurate measurement of a target nucleic acid concentration in the solution to be detected. The presence or absence or degree of aggregation can also be detected by aggregated state detection methods such as microscopic observation, the detection of light scattered from particles, electrochemical detection, surface plasmon resonance, magnetic measurement and quartz crystal microbalance.

<Nucleic Acid Type>

A target nucleic acid to which the detection method of the present invention can be applied is not particularly limited as long as the nucleic acid has a base sequence for which primers for gene amplification reaction can be designed and gene amplification reaction using the primers can be practiced. Such a nucleic acid that can be utilized may be any of DNA and RNA and may be any of single-strand and double-strand nucleic acids. The base sequence of such a nucleic acid that can be used includes at least a portion of a gene sequence to be detected. The presence or absence or amount of the nucleic acid including such a sequence can be detected to thereby examine the presence or absence or copy number of the gene. The nucleic acid is not limited by origin. Specifically, any of natural nucleic acids (e.g., animal-, plant-, microorganism- and virus-derived nucleic acids) and artificially synthesized nucleic acids (e.g., chemically synthesized nucleic acids and nucleic acids synthesized in a gene engineering manner) can be utilized as a target nucleic acid. In the present specification, a base sequence for expressing some function is referred to as a gene. Thus, nucleic acids (DNA and RNA) and genes do not necessarily have the relationship of higher/lower conception.

A sample to which the detection method of the present invention can be applied is not particularly limited as long as the sample possibly contains a target nucleic acid. Examples thereof can include biological samples and environment-derived samples. Examples of the biological samples can include animal body fluids (e.g., blood, serum, plasma, spinal fluids, sweat, saliva, urine and seminal fluids), hair, excrement, organs and tissues, animals and plants themselves and dry products thereof. Examples of the environment-derived samples can include river water, lake water, sea water and soils. When these samples are in a liquid state, these samples can be used as an analyte solution. These sample solutions may be subjected to dilution, separation and purification, if necessary. Alternatively, when these samples are in a solid state, eluates of the samples can be used as an analyte solution. These sample solutions may be subjected to dilution, separation and purification, if necessary.

<Nucleic Acid Probe>

A nucleic acid probe used in the present invention may be any substance that can be associated sequence-specifically with a nucleic acid and does not bind to a nucleic acid-aggregating agent. Examples of the nucleic acid probe that is preferably used include peptide nucleic acids (PNAs). The chain length of the nucleic acid probe can be 18 mer or shorter from the viewpoint of easy synthesis. Moreover, the sequence of the nucleic acid probe can be determined appropriately according to the sequence of the target nucleic acid. The peptide nucleic acid does not have a phosphodiester bond in its structure. Specifically, the peptide nucleic acid is not a “nucleic acid” species, though its name has the term “nucleic acid”.

When peptide nucleic acids are used as the nucleic acids of labeled nucleic acid probes, the single-base mutation of the target nucleic acid can also be detected. This is attributed to the high recognizing ability of the peptide nucleic acids. Because of the high recognizing ability of the peptide nucleic acids, the labeled nucleic acid probes do not form an associate with a target nucleic acid having single-base mutation. Thus, in such a case, aggregation does not occur. On the other hand, the labeled nucleic acid probes form an associate with a target nucleic acid having no mutation and are therefore aggregated, as described above. Accordingly, a target nucleic acid having no mutation can be distinguished from a target nucleic acid having single-base mutation (i.e., single-base mutation can be detected). Any of base substitution, insertion and deletion can be detected as such single-base mutation. In this context, the “single-base mutation of a target nucleic acid” means that one of bases of the target nucleic acid is substituted or deleted or that one base is inserted (added) to bases of the target nucleic acid.

<Microcarrier>

A microcarrier used in the present invention may be any minute substance that can be immobilized on a nucleic acid probe and does not hinder the aggregation reaction of the present approach. For example, colloidal gold, colloidal silver, metal microstructures (e.g., gold and silver nanorods), magnetic beads, polystyrene beads and glass beads can be used as microcarriers. Any size of the microcarrier according to the present invention can be utilized as long as the size does not hinder the aggregation reaction of the present approach. The microcarriers is preferred to be within the range of 5 nm to 5000 nm in average diameter.

The nucleic acid probes can be immobilized onto the microcarrier surfaces by use of a known oligonucleotide immobilization method. Examples of the immobilization method, for example, for colloidal gold microcarriers, include a method utilizing the covalent bond of a sulfur atom in organic matter having a sulfur atom with the surface of a metal such as gold. In this case, nucleic acid probes in which a thiol group is introduced in advance can be mixed with colloidal gold particles to thereby immobilize the nucleic acid probes on the colloidal gold particles. Alternatively, the microcarrier surfaces can be aminated, carboxylated or thiolated in advance with a silane agent or the like to thereby immobilize the nucleic acid probes onto the microcarrier surfaces through an adsorption method or coupling reaction. An alternative method that can be used includes adsorbing streptavidin in advance onto the microcarriers and immobilizing biotin-modified nucleic acid probes thereon through the selective binding of streptavidin to biotin.

<Nucleic Acid-Aggregating Agent>

A nucleic acid-aggregating agent used in the present invention is not particularly limited as long as the nucleic acid-aggregating agent is a substance that has the property of binding to nucleic acids, regardless of single-strand or double-strand nucleic acids, and aggregating the nucleic acids. Examples of the nucleic acid-aggregating agent can include Hoechst 33258 [(Hoechst); 2′-(4-hydroxyphenyl)-5-(4-methyl-1-piperazinyl)-2,5′-bi-1H-benzimidazole], Hoechst 33342 [(Hoechst); 2′-(4-ethoxyphenyl)-5-(4-methyl-1-piperazinyl)-2,5′-bi-1H-benzimidazole], nuclear yellow [(SIGMA); 4-[5-(4-methyl-1-piperazinyl)[2,5′-bi-1H-benzimidazol]-2′-yl]-benzenesulfonamide] and mitoxantrone [(SIGMA); 1,4-dihydroxy-5,8-bis{2-[(2-hydroxyethyl)-amino]ethyl}aminoanthracene-9,10-dione]. These nucleic acid-aggregating agents do not bind to PNA having no phosphodiester bond.

In general, a substance more suitable as a nucleic acid-aggregating agent binds to an associate between the target nucleic acid and the nucleic acid probe but does not bind to the nucleic acid probe itself (does not bind to the nucleic acid probe present in an state unbound with the nucleic acid). Thus, the combination of the nucleic acid probes with the nucleic acid-aggregating agent can be selected arbitrarily as long as the combination satisfies such conditions.

<Examination Kit>

Next, an exemplary embodiment of the examination kit of the present invention will be described

The examination kit as a second embodiment of the present invention includes at least an examination reagent. The examination kit of the present invention may include only single or plural examination reagents or may include an examination container and so on in addition to the examination reagent(s). The examination reagent has at least the labeled nucleic acid probes 10 including the nucleic acid probes 1 and the microcarriers 9.

The examination reagents included in the exemplary embodiment of the examination kit of the present invention include the labeled nucleic acid probes 10 themselves including the nucleic acid probes 1 and the microcarriers 9 or a reagent having the labeled nucleic acid probes 10 and the nucleic acid-aggregating agent 6 itself or a reagent having this nucleic acid-aggregating agent 6. These reagents may be in a dry state or in a solution state and may be supplemented with a reaction-promoting reagent, a surfactant, and the like.

For using the examination kit, the examination reagents (the labeled nucleic acid probes 10 themselves or a reagent having the labeled nucleic acid probes 10 and the nucleic acid-aggregating agent 6 itself or a reagent having this nucleic acid-aggregating agent 6) are added in this order to an analyte solution to prepare a mixed solution. The presence or absence or degree of aggregation of the microcarriers occurring depending on the presence or absence of the target nucleic acid in the mixed solution may be measured. The reagents are added in this order because the reaction of the nucleic acid-aggregating agent with the nucleic acid is significantly faster than the reaction of the nucleic acid-aggregating agent with the associate. On the other hand, for example in case that these reactions may hardly differ in speed, the reaction of the unassociated target nucleic acid with the aggregating agent may not matter. In this case, the labeled nucleic acid probes and the nucleic acid-aggregating agent can be added simultaneously to the analyte solution. In such a case, a reagent having the labeled nucleic acid probes and also having the nucleic acid-aggregating agent may be prepared and added to the analyte solution.

The use of this kit enables rapid and convenient detection of a target nucleic acid.

### EXAMPLES

Hereinafter, Examples of the present invention will be described.

### Example 1

### Preparation of Labeled Nucleic Acid Probe Solution

A colloidal gold solution stored under refrigeration, which contains colloidal gold particles of 15 nm in diameter dispersed as microcarriers, is warmed from the refrigeration temperature to room temperature. A 0.9 ml aliquot of this solution (manufactured by BB International) is mixed with 0.1 ml of 50 mM KH₂PO₄ to prepare a colloidal gold solution a.

Next, 3 nmol of peptide nucleic acids (PNAs) having cysteine at the N terminus (NH₂-Cys-O—O-CTCCTCTTGACCTGC-H) (manufactured by Greiner Japan) is mixed with the colloidal gold solution a. This mixture is reacted overnight at room temperature to obtain a mixed solution b. Next, the mixed solution b is centrifuged at 14000 rpm for 30 minutes. The supernatant is removed, and 1 ml of 50 mM KH₂PO₄ is newly added to the residue. After additional centrifugation operation under the same condition, this precipitate is used as a labeled nucleic acid probe solution.

<Detection Reaction>

A 10 μl aliquot of target nucleic acid (5′-ACAGCAGGTCAAGAGGAGTA-3′) solution (manufactured by Greiner Japan) is added to 5 μl of the prepared labeled nucleic acid probe solution to associate the target nucleic acids with the probes. Next, aggregation is promoted by adding 5 μl of 400 μM Hoechst 33258 (manufactured by Wako Pure Chemical Industries) as a nucleic acid-aggregating agent.

When Hoechst 33258 is added to nucleic acid probes unsupplemented with target nucleic acids, this solution does not exhibit a color change. On the other hand, when Hoechst 33258 is added to nucleic acid probes supplemented and associated with target nucleic acids, this solution turns purple due to the aggregating effect of Hoechst 33258. This color change is caused because Hoechst 33258 aggregates target nucleic acids through binding and therefore aggregates colloidal gold particles bound with the nucleic acid probes associated with the target nucleic acids. Therefore, according to the present Example, the presence or absence of a target nucleic acid can be detected conveniently.

### Example 2

### Preparation of Target Nucleic Acid

PCR is performed using Human Genomic DNA (manufactured by Novagen) as an examination sample. Human Apolipoprotein E-encoding genes are selected as target nucleic acids.

PCR is performed using a PCR amplification kit (manufactured by ToYoBo) under the following conditions: Human Genomic DNA (0.5 μl), 10× Buffer (2.5 μl), 2 mM dNTP (2.5 μl), 25 mM MgSO₄ (1.5 μl), KOD (0.5 μl), F-Primer (1.5 μl), R-Primer (1.5 μl) and H₂O (14.5 μl) are mixed and subjected to 35 temperature cycles involving 95° C. for 15 seconds and 68° C. for 15 seconds.

Then, the single-strand nucleic acids are amplified by asymmetric PCR. The resulting PCR products are used as examination samples.

(Preparation of Nucleic Acid Probe>

Labeled nucleic acid probe solutions having nucleic acid probes shown in Table 1 are prepared in the same way as in Example 1 except that the peptide nucleic acids (PNAs) used have base sequences shown in Table 1.

TABLE 1

| | |
|---|---|
| | Color |
| | change of |
| Sequence | solution |

| | | |
|---|---|---|
| 112TGC | H-CCTCCTGCACACGCCGGC-O-O-Cys-NH₂ | Present |
| 112CGC | H-CCTCCTGCACGCGCCGGC-O-O-Cys-NH₂ | Absent |
| 158TGC | H-ACGTCTTCACGGACCGTC-O-O-Cys-NH₂ | Absent |
| 158CGC | H-ACGTCTTCGCGGACCGTC-O-O-Cys-NH₂ | Present |

These nucleic acid probes are as follows:

112TGC is a peptide nucleic acid complementary to a portion of a gene encoding Human Apolipoprotein E having cysteine as the 112th amino acid.

112CGC is a peptide nucleic acid complementary to a portion of a gene encoding Human Apolipoprotein E having arginine as the 112th amino acid.

158TGC is a peptide nucleic acid complementary to a portion of a gene encoding Human Apolipoprotein E having cysteine as the 158th amino acid.

158CGC is a peptide nucleic acid complementary to a portion of a gene encoding Human Apolipoprotein E having arginine as the 158th amino acid.

<Detection Reaction>

A 10 μl aliquot of the prepared PCR product (target nucleic acid) solution is added to 5 μl each of the labeled nucleic acid probe solutions having the respective nucleic acid probes shown in Table 1 to perform association reaction. Next, 5 μl of 400 μM Hoechst 33258 (Wako Pure Chemical Industries) is added as a nucleic acid-aggregating agent to each of the solutions. The presence or absence of aggregation reaction is examined.

As shown in Table 1, the labeled nucleic acid probe solution having 112TGC and the labeled nucleic acid probe solution having 158CGC exhibit a color change, whereas the labeled nucleic acid probe solution having 112CGC and the labeled nucleic acid probe solution having 158TGC do not exhibit a color change.

This means that the target nucleic acid in the sample is a Human Apolipoprotein E-encoding gene having T as a base at a single-base substitution site corresponding to the 112th position in the amino acid sequence and C as a base at a single-base substitution site corresponding to the 158th position in the amino acid sequence. Specifically, this indicates that the 112th and 158th amino acids are cysteine and arginine, respectively. A gene having this combination is called ε-3 type, which encodes Human Apolipoprotein E3. Alternatively, ε-4 type, which has been known to serve as a very strong risk factor for Alzheimer's disease and cardiovascular disease, has arginine at both the 112th and 158th positions.

Thus, according to the present Example, the presence or absence of single-base substitution can also be detected conveniently.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2006-266538, filed Sep. 29, 2006, which is hereby incorporated by reference herein in its entirety.

What is claimed is:

1. A nucleic acid detection method for detecting a target nucleic acid in an analyte solution, including the steps of:
preparing a mixed solution by mixing: labeled nucleic acid probes in which nucleic acid probes to be associated with the target nucleic acid are immobilized on microcarriers; the analyte solution; and a nucleic acid-aggregating agent; and
detecting the presence or absence or degree of aggregation of the microcarriers in the mixed solution.

2. The nucleic acid detection method according to claim 1, wherein the step of preparing a mixed solution by mixing: labeled nucleic acid probes in which nucleic acid probes to be associated with the target nucleic acid are immobilized on microcarriers; the analyte solution; and a nucleic acid-aggregating agent is
a step of preparing a mixed solution by mixing the labeled nucleic acid probes with the analyte solution and then adding the nucleic acid-aggregating agent thereto.

3. The nucleic acid detection method according to claim 1, wherein the step of preparing a mixed solution by mixing: labeled nucleic acid probes in which nucleic acid probes to be associated with the target nucleic acid are immobilized on microcarriers; the analyte solution; and a nucleic acid-aggregating agent is
a step of simultaneously mixing the labeled nucleic acid probes, the analyte solution and the nucleic acid-aggregating agent.

4. The nucleic acid detection method according to claim 1, wherein the nucleic acid probes are peptide nucleic acids.

5. The nucleic acid detection method according to claim 2, wherein the nucleic acid probes are peptide nucleic acids.

6. The nucleic acid detection method according to claim 3, wherein the nucleic acid probes are peptide nucleic acids.

7. The nucleic acid detection method according to claim 1, wherein the step of detecting the presence or absence or degree of aggregation of the microcarriers is performed by measuring a color change of the mixed solution.

8. The nucleic acid detection method according to claim 2, wherein the step of detecting the presence or absence or degree of aggregation of the microcarriers is performed by measuring a color change of the mixed solution.

9. The nucleic acid detection method according to claim 3, wherein the step of detecting the presence or absence or degree of aggregation of the microcarriers is performed by measuring a color change of the mixed solution.

10. The nucleic acid detection method according to claim 1, wherein the nucleic acid-aggregating agent is a substance that does not bind to the nucleic acid probe itself and binds to an associate between the target nucleic acid and the labeled nucleic acid probe.

11. The nucleic acid detection method according to claim 2, wherein the nucleic acid-aggregating agent is a substance that does not bind to the nucleic acid probe itself and binds to an associate between the target nucleic acid and the labeled nucleic acid probe.

12. The nucleic acid detection method according to claim 3, wherein the nucleic acid-aggregating agent is a substance that does not bind to the nucleic acid probe itself and binds to an associate between the target nucleic acid and the labeled nucleic acid probe.

13. An examination kit for detecting a target nucleic acid in an analyte solution, including at least one examination reagent,
the one examination reagent including labeled nucleic acid probes in which nucleic acid probes are immobilized on microcarriers.

14. The examination kit according to claim 13, further including an examination reagent including a nucleic acid-aggregating agent.


SEQUENCE LISTING (1 sequence)

SEQ ID NO: 1 - DNA, 20 bp, Artificial
  target nucleic acid
acagcaggtcaagaggagta